(19) United States
(12) Patent Application Publication
(10) Pub. No.: US 2010/0099974 A1
(43) Pub. Date: Apr. 22, 2010

(54) System for Generating a Multi-Modality Imaging Examination Report

(75) Inventor: Ravindranath S. Desai, Chelsea, MI (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvem, PA (US)

(21) Appl. No.: 12/509,042

(22) Filed: Jul. 24, 2009

Related U.S. Application Data
(60) Provisional application No. 61/106,635, filed on Oct. 20, 2008.

(51) Int. Cl. A61B 5/055; G06Q 50/00; G06F 17/30; G06N 5/02; G06F 7/14

(52) U.S. Cl. 600/411; 705/3; 706/47; 707/E17.019; 707/E17.044; 707/708

(57) ABSTRACT

A system processes medical report data associated with different types of imaging modality devices to provide a composite examination report. An acquisition processor in the system acquires multi-modality medical imaging examination report data for examinations performed on a patient by different types of imaging modality device. A report processor processes acquired multi-modality medical imaging examination report data items by, in response to predetermined selection riles, selecting between individual data items in the acquired multi-modality medical imaging examination report data items to provide a single individual data item for incorporation in a composite report. The report processor maps individual data items including the single individual data item in the acquired multi-modality medical imaging examination report data items to corresponding data fields in a composite report data structure in memory in response to predetermined mapping information. An output processor outputs data representing the composite report to a destination device.

This is a non-provisional application of provisional application Ser. No. 61/106,635 filed Oct. 20, 2008, by Ravindranath. S. Desai.

### FIELD OF THE INVENTION

This invention concerns a system for processing medical report data associated with different types of imaging modality devices to provide a composite examination report.

### BACKGROUND OF THE INVENTION

A multi-modality workflow (e.g., for one or more of magnetic resonance (MR), computerized tomography (CT) scan, X-ray, Ultra Sound imaging systems) comprises multiple tasks that may individually produce one or more evidence documents (ED). An ED contains clinical data relating to a specific task that generated it. It is typical in known systems for individual modality tasks to produce their own reports (or set of reports). Data points in a modality task are individually reported and a physician needs to manually reconcile data points from individual modalities that may be in conflict. This is particularly needed for demographic information, where each task or modality system may obtain patient information from different sources.

Known systems produce and maintain individual independent task or modality system reports and a physician manually collates data from multiple tasks or modalities to support examination and diagnosis. This increases the likelihood of errors. In known systems, collating such data involves laborious, time consuming manual effort. A system according to invention principles addresses these deficiencies and related problems.

### SUMMARY OF THE INVENTION

The inventors have advantageously recognized that it is desirable to automatically generate a report targeted to a particular audience (patient, referring physician, nurse, for example) that contains information from evidence documents from multiple different imaging modality systems. A system consolidates data from clinical reports and structured report documents and other data sources into a composite examination report by resolving data conflict and adaptively generating single examination report or multiple reports. A system processes medical report data associated with different types of imaging modality devices to provide a composite examination report. An acquisition processor in the system acquires multi-modality medical imaging examination report data for examinations performed on a patient by different types of imaging modality device. A report processor processes acquired multi-modality medical imaging examination report data items by, in response to predetermined selection rules, selecting between individual data items in the acquired multi-modality medical imaging examination report data items to provide a single individual data item for incorporation in a composite report. The report processor maps individual data items including the single individual data item in the acquired multi-modality medical imaging examination report data items to corresponding data fields in a composite report data structure in memory in response to predetermined mapping information. An output processor outputs data representing the composite report to a destination device.

### BRIEF DESCRIPTION OF THE DRAWING

FIG. 1 shows a system for processing medical report data associated with different types of imaging modality devices to provide a composite examination report, according to invention principles.

FIG. 2 shows a common data model service system for processing report documents acquired from multi-modality imaging task flows and other data from different sources, according to invention principles.

FIG. 3 shows a multi-modality imaging task flow and processing of report documents associated with the task flow by a common data model service, according to invention principles.

FIG. 4 shows a process for resolving conflicts in consolidating data items from multi-modality imaging report documents to provide a composite report, according to invention principles.

FIG. 5 illustrates a DICOM SR compatible clinical finding change document of an MR report.

FIG. 6 illustrates a document showing a change data set of an MR report compatible with the common data model converted from the DICOM SR compatible clinical finding change document of FIG. 5, according to invention principles.

FIG. 7 illustrates status of the Common Data Service function after receiving MR report data concerning two tumors, according to invention principles.

FIG. 8 illustrates status of the Common Data Service function after receiving MR and CT report data concerning two tumors, according to invention principles.

FIG. 9 shows a flowchart of a process performed by a system for processing medical report data associated with different types of imaging modality devices to provide a composite examination report, according to invention principles.

### DETAILED DESCRIPTION OF THE INVENTION

A system automatically produces reports that contain data that is merged from multiple tasks associated with different imaging modality systems (e.g., for one or more of magnetic resonance (MR), computerized tomography (CT) scan, X-ray, Ultra Sound imaging systems), in a multi-modality imaging device workflow. System report templates address different multi-modality workflows, so the system presents a diagnosing physician with a reasonable default set of reports accommodating data expected to be generated in a workflow (task sequence). The system automates consolidation of data from different tasks in a multi-modality workflow into a single composite report. An evidence document (ED) associated with an imaging modality system task contains clinical data relating to a specific task that generated it. The inventor has advantageously recognized it is desirable for a composite report at the end of a workflow to contain amalgamated results from the evidence documents from multiple different imaging modality system associated tasks and the composite report reflects a single approved value for clinical and other data. This addresses deficiencies of known systems which lack a comprehensive, consistent way to have a single reporting system access data from different modalities and combine the data in a single report.

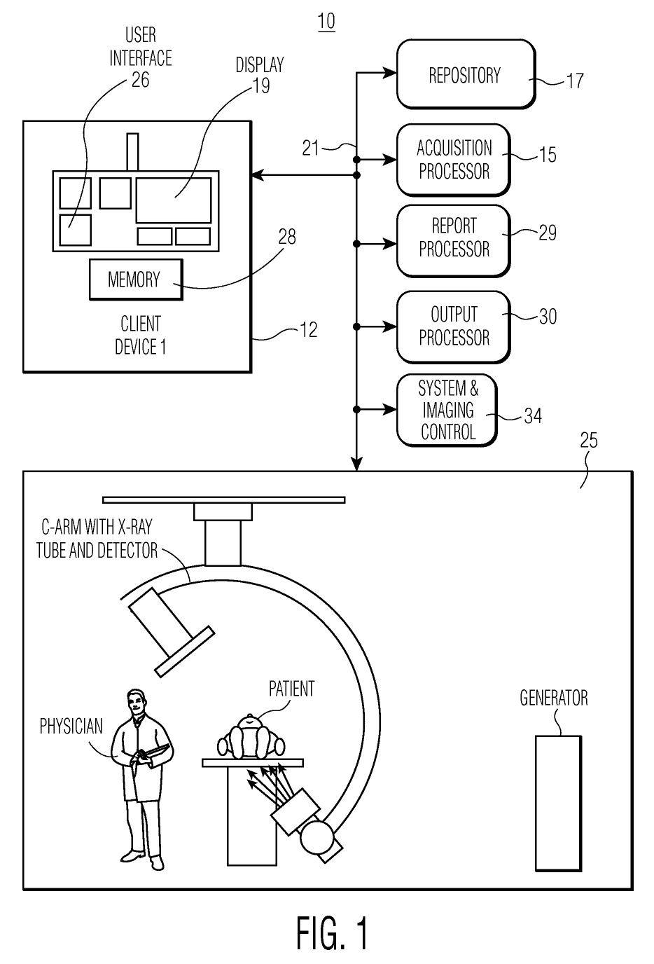

FIG. 1 shows system 10 for processing medical report data associated with different types of imaging modality devices to provide a composite examination report. System 10 includes one or more processing devices (e.g., workstations, computers or portable devices such as notebooks, Personal Digital Assistants, phones) 12 that individually include memory 28, a user interface 26 enabling user interaction with a Graphical User Interface (GUI) and display 19 supporting GUI and image presentation in response to predetermined user (e.g., physician) specific preferences. As well as device 12, system 10 also includes different multi-modality imaging devices 25, repository 17, acquisition processor 15, report processor 29, output processor 30 and system and imaging controller 34 intercommunicating via network 21. Imaging modality devices 25, although shown as a single X-ray imaging device in FIG. 1, includes at least two different device types of, (a) an MR imaging device, (b) a CT scan imaging device, (c) an X-ray imaging device, (d) an Ultra-sound imaging device and (e) a nuclear PET scanning imaging device. Display 19 of processing device 12 presents display images comprising a GUI. At least one repository 17 stores multi-modality medical image studies for patients in DICOM (Digital Imaging and Communications in Medicine) standard compatible (or other) data format. A medical image study individually includes multiple image series of a patient anatomical portion which in turn individually include multiple images and sometimes DICOM structured reports.

Acquisition processor 15 acquires multi-modality medical imaging examination report data for examinations performed on a patient by different types of imaging modality devices 25. Report processor 29 processes acquired multi-modality medical imaging examination report data items by selecting between individual data items in the acquired multi-modality medical imaging examination report data items to provide a single individual data item for incorporation in a composite report, in response to predetermined selection rules. Report processor 29 maps individual data items including the single individual data item in the acquired multi-modality medical imaging examination report data items to corresponding data fields in a composite report data structure in memory (e.g., repository 17) in response to predetermined mapping information. Output processor 30 outputs data representing the composite report to a destination device (e.g., display 19).

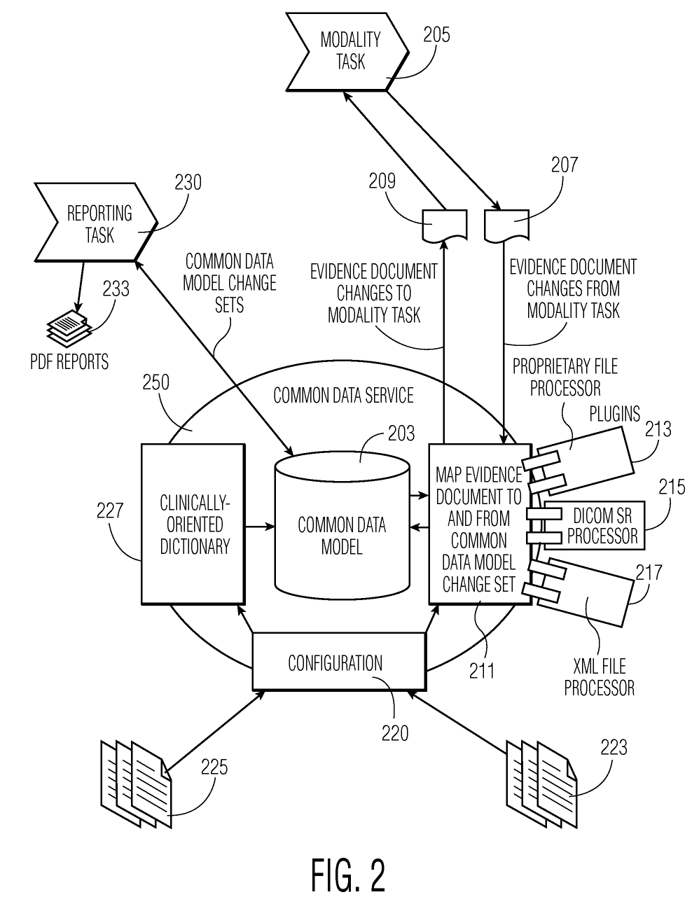

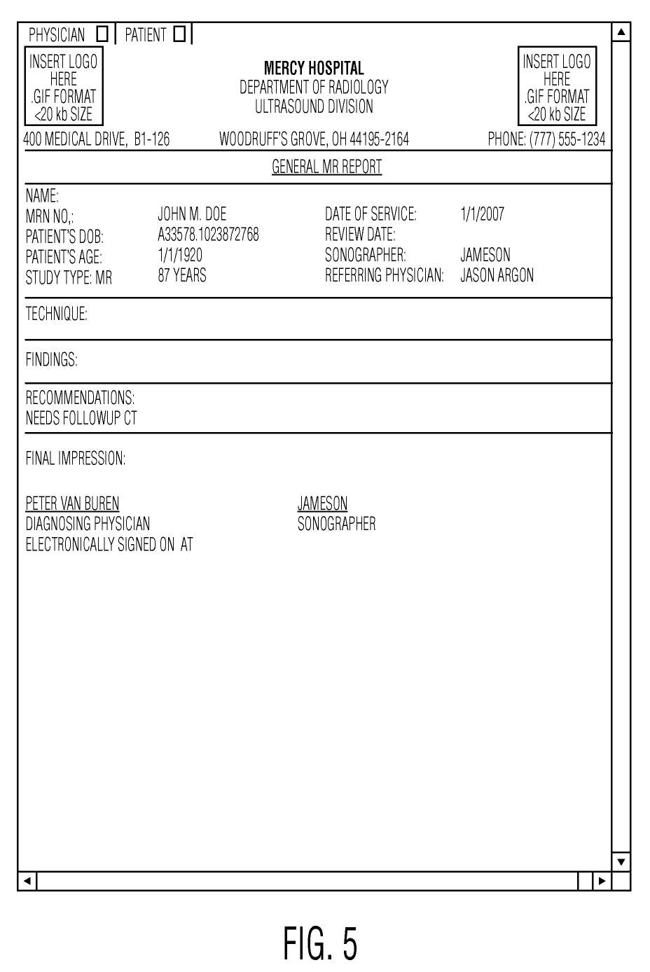

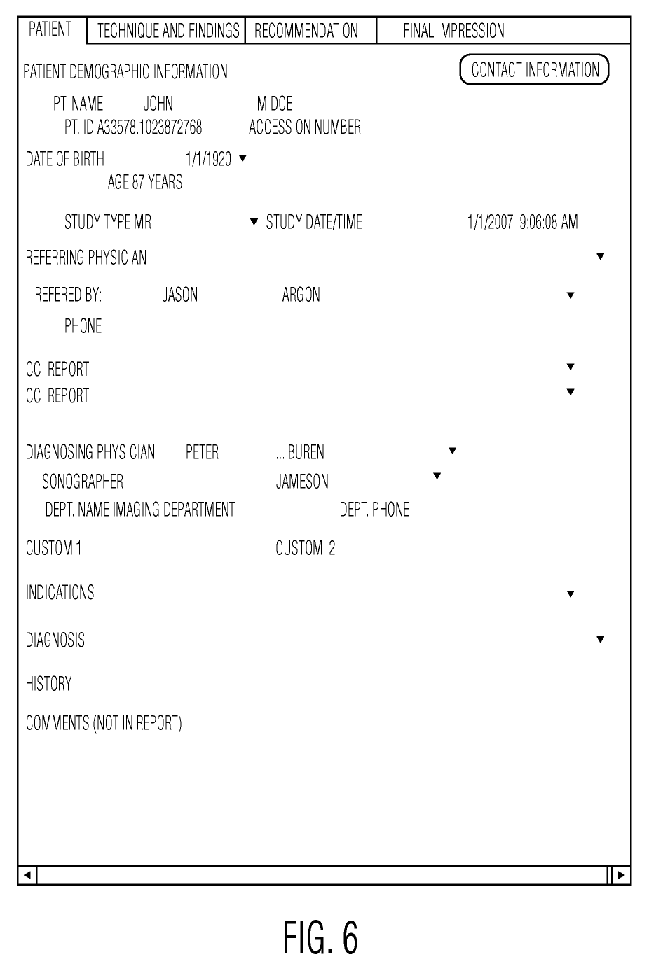

FIG. 2 shows a common data model service system 250 employed by acquisition processor 15 and report processor 29 (FIG. 1) for processing report documents acquired from multi-modality imaging task flows and other data from different sources. Common data model service system 250 includes data model 203, configuration processor 220, a clinical dictionary 227 of items that can be placed in data model 203 and mapping processor 211 that maps an evidence document both to and from, a common data model compatible data change set. Mapping processor 211 may comprise a proprietary file processor 213, DICOM SR (Structured Report) processor 215 and XML file processor 217 and another processor (not shown). System 250 determines which mapping processor to employ in response to Meta-data conveyed together with, or within, an evidence document change data set. Mapping processor 211 maps an evidence document both to and from, a common data model compatible data change set using data items including codes, terms and identifiers in dictionary 227 that may be incorporated in the hierarchical common data model 203. FIG. 5 illustrates a DICOM SR compatible clinical finding change document comprising an MR imaging report converted by DICOM SR processor 215 in mapping processor 211, to the document of FIG. 6 compatible with common data model 203. Specifically, FIG. 6 shows a change data set of an MR report compatible with common data model 203 converted by processor 215 from the DICOM SR compatible clinical finding change document of FIG. 5.

Configuration processor 220 configures dictionary 227 and mapping processor 211 with predetermined definitions (e.g., medical term definitions and synonyms) 225 and DICOM SR compatible mapping configuration files 223 individually including template identifier and modality type identifier and predetermined data item conflict resolution and selection rules. Common data service system 250 initiates operation by acquiring configuration files 223 and definitions 225 and configures and initializes dictionary 227 and mapping processor 211. Specifically, configuration processor 220 configures mapping processor 211 with available different evidence document type file processors including processors 213, 215 and 217. A modality expert interacts with imaging modality system 25 in performing task 205 to produce a set of evidence documents 207 (e.g., in the form of DICOM Structured Reports). The modality expert encodes a mapping 211 of data items in individual evidence documents into clinically relevant common reporting data model 203. This includes encoding rules for automated conflict resolution. Clinically relevant common reporting data model 203 is an extensible, hierarchical, model that contains text, measurement, observation, Boolean, date/time, DICOM, demographic (age, gender, height, weight), other personal data and image data in a wide variety of formats including DICOM and binary. This data is represented hierarchically by the use of context information and multi-keyed data tables. A change data set from common data service system 250 are concurrently provided to reporting task 230 and mapped to corresponding evidence documents 209 that are transmitted to associated task 205 being performed in a multi-modality imaging workflow using an executable application. A diagnosing physician initiates reporting task 230 which accesses common reporting data model 203 and interacts with it. A reporting system executing reporting task 230 produces both worksheets and reports 233 that operate on data in model 203.

A clinical expert familiar with a multi-modality task workflow employs system 250 to produce worksheets and reports 233 that are capable of interacting with and displaying, data derived from model 203. The common reporting data model 203 and worksheets and reports 233 are accessible via a local or remote diagnosing physician workstation (e.g., via a network such as the Internet). Evidence document templates and common reporting data model mapping file processors 213, 215 and 217 are stored in a processing device such as a computer or server. In response to a workflow being started at a diagnosing physician workstation such as client device 12 (FIG. 1), modality tasks and an associated executable application involve collecting data and transmitting the collected data to system 10 in the form of evidence documents. System 10 receives the evidence documents, maps them to common reporting data model 203, and automatically resolves conflicts with data already in the model.

System 10 initiates a task assigned to an imaging modality expert that requires the expert to understand information contained in evidence documents produced in an imaging system and map the information into a common data model 203 (FIG. 2). Common data model 203 is a superset of supported task and imaging modality system related data and enables an imaging modality system expert to present structured report data produced during a task involving interaction with the expert in a clinically relevant way. Imaging modality tasks continue to produce evidence documents that contain the data related to respective individual tasks. In one embodiment the evidence documents are transmitted to units 15 and 29 that acquire data contained in the documents and maps the data to common data model 203 within a reporting system. If there are data conflicts that can be resolved by an automated rule (e.g., Molecular Imaging (MI) image data may be viewed as more reliable than computerized Tomography (CT) data for certain measurements), report processor 29 applies the rule during the mapping. Other conflicts may require interaction by a clinical specialist. Common data service system 250 automatically identifies conflicts to be identified to a user at client device 12 to be resolved manually.

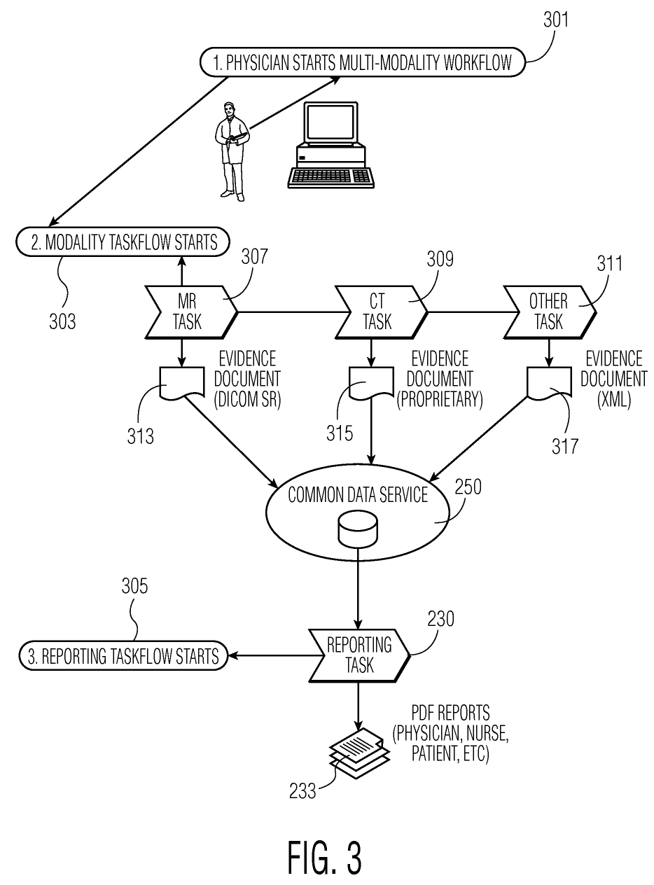

FIG. 3 shows a multi-modality imaging task flow and processing of report documents associated with the task flow by common data model service system 250. In operation, a physician in step 301 starts a multi-modality imaging workflow. A physician in step 303 starts a multi-modality task sequence with an MR imaging related task 307, CT imaging related task 309 and another imaging modality task 311 producing evidence documents in DICOM SR 313, proprietary 315 and XML 317 formats respectively and providing the documents to common data service system 250 and reporting task 230. In an example of operation, a physician tracks progress of a tumor in a patient lung and uses both CT imaging and MR imaging on a patient to obtain different views of the tumour. Three measurements of the tumour are shape, diameter, and volume. Shape is recorded as either being elongated, spherical, or erratic. Length is measured in millimeters, and volume in cubic millimeters.

In response to evidence document data from different tasks associated with different types of imaging modality system being filtered and provided to single common data model 203, the system initiates a reporting task sequence 230 in step 305. The system produces reports 233 targeted to a particular audience which can contain data from multiple tasks associated with multiple different types of imaging modality system. A physician advantageously has the ability of editing data sent from an imaging system for incorporation in a report. The system ensures that new evidence document data sent during an imaging modality task does not overwrite data entered by a physician. Common data model 203 tracks and applies prioritized rules to resolve conflicts in attempted data changes and maintains an audit trail identifying changes made, when changes are made, by who and source and destination of changes.

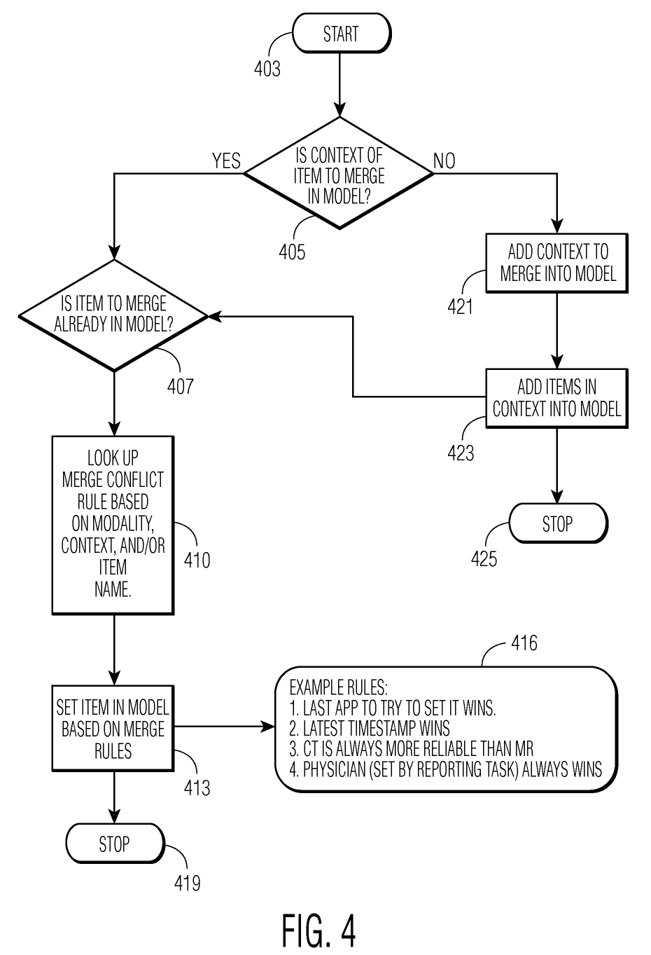

FIG. 4 shows a process performed by common data model service system 250 for resolving conflicts in consolidating data items from multi-modality imaging report documents to provide a composite report employed by common data model service system 250 (FIG. 2). In step 405 following the start at step 403, system 250 determines whether context information of a data item to be merged into data model 203 is already in model 203. If the context information is not already in model 203, the context information is identified to be merged in step 421. If the context information is already in model 203, the process continues with step 407. In step 423, it is determined if the data item associated with the added context information is to be added to model 203 without conflict checks. If no conflict check is to be applied, the data item is added in appropriate context to model 203 in step 423 and the process ends at step 425. In step 423 if it is determined that a conflict check is to be performed, the process continues with step 407.

In step 407, common data model service system 250 determines if a value of the data item to be merged into model 203 is already present in model 203 and that there is a conflict. If there is no conflict, the data item value is added to model 203 in step 413 and the process ends in step 419. If it is determined in step 407 that there is conflict and a value of the data item already exists in model 203, system 250 in step 410 looks up conflict rules to be applied to govern merging the data item into model 203 based on imaging modality device type providing the data item, context information and the data item name. In step 413, system 250 resolves a conflict between data items derived using different types of imaging modality device by selecting a data item value to add to model 203 in response to rules 416 and associated rule priority, identified in step 410. For example, rules may determine, in priority order (a later applied rule being of higher priority superseding previous rules), that 1) a last executable application to attempt to select a particular data item to be used wins, 2) that a latest data item value time stamp (i.e., the latest acquired data item) wins, 3) that a CT imaging device derived data item takes precedence over an MR device derived data item and 4) a data item selected by a physician in a reporting task wins. The process of FIG. 4 ends at step 419.

In an example of operation, system 250 (FIG. 2) determines for a particular clinical type of case and diagnosis, that if there is a data conflict between the same image parameter measurement made using different images acquired using corresponding different types of imaging modality device, the latest data measurement is used. Except, however, in the case of a Tumor length measurement, system 250 gives precedence to, and uses, an MR derived value since it is assumed an MR imaging device produces a more accurate Tumor length measurement value than a CT, X-ray, or Ultrasound device, for example. However, in the case of a tumor volume measurement, system 250 determines that a volume measurement derived by averaging tumor volume measurements provided by different types of imaging modality systems is used. Further, in the case of a tumor shape determination, configuration processor 220 indicates that a tumor shape is determined using a CT imaging modality system since tumor shape is clearer in a CT image. Therefore, if a CT image of tumor shape is available it is used and included in a final report.

Common Data Service system 250 employs the conflict rules in Table I for resolving conflicts between data items derived by different types of imaging modality system.

TABLE I

| Modality | Context | Measurement | Conflict Resolution Strategy |
|---|---|---|---|
| <any> | <any> | <any> | Latest Wins |
| MR | Tumor | Length | Always Wins |
| <any> | Tumor | Volume | Contributes to Average |
| CT | Tumor | Shape | Always Wins |

Other hierarchically prioritized rules may also be used to resolve the conflicts.

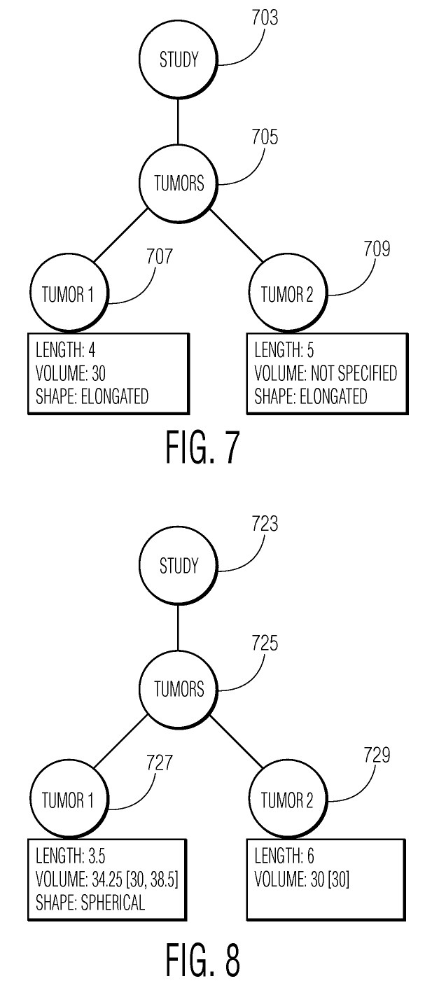

FIG. 7 illustrates status of the Common Data Service system 250 after receiving MR report data concerning two tumors. An MR imaging study is acquired in step 703 and tumor measurements of Table II are made in step 705 of a first tumor (tumor 1) 707 and a second tumor (tumor 2) 709. The MR imaging study acquisition task is performed interactively in response to physician commands and captures findings to be included in a report. The measurement findings include those shown in Table II, are transmitted to Common Data Service system 250 for inclusion in common data model 203.

TABLE II

| Context | ID | Measurement | Value |
|---|---|---|---|
| Tumor | 1 | Length | 4 mm |
| Tumor | 1 | Volume | 30 mm³ |
| Tumor | 1 | Shape | Elongated |
| Tumor | 2 | Length | 5 mm |
| Tumor | 2 | Shape | Elongated |

In this example, an MR task is performed providing report data concerning two tumors (having identifiers ID 1 and ID 2). The first tumor is 4 mm long with an apparent volume of 30 mm³, and an elongated shape. The second tumor is 5 mm long with an elongated shape, but no volume measurement is available for the second tumor.

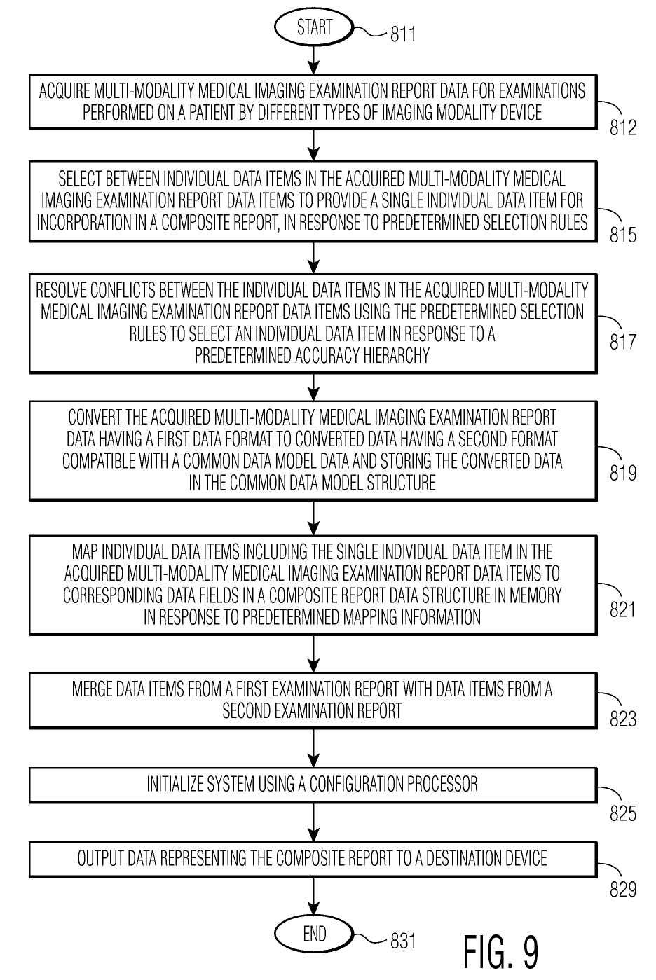

FIG. 8 illustrates status of the Common Data Service function after receiving MR and CT report data concerning the two tumors of FIG. 7. A CT imaging study is acquired in step 723 and tumor measurements of Table III are made in step 725 of the first tumor (tumor 1) 727 and the second tumor (tumor 2) 729.

TABLE III

| Context | ID | Measurement | Value |
|---|---|---|---|
| Tumor | 1 | Length | 3.5 mm |
| Tumor | 1 | Volume | 38.5 mm³ |
| Tumor | 1 | Shape | Spherical |
| Tumor | 2 | Volume | 30 mm³ |
| Tumor | 2 | Length | 6 mm |

The CT imaging task measurements indicate a slightly smaller length, but larger overall volume, and spherical shape for the first tumor. The CT imaging task measurements indicate a 30 mm³ volume, 6 mm length, but no shape information for the second tumor. In response to completion of the CT task, findings are sent to Common Data Service system 250 which resolves data conflicts. Since MR length is used, the CT lengths are ignored. Likewise, since CT Shape is used, the shape for Tumor 1 is set to Spherical. Further, since no shape was recorded on the CT task for Tumor 2, the value recorded on the MR task (elongated) is left in place. In this case, both the CT and MR tasks contribute to the derived volume value by both creating measurement instances and having the derived value be the average of the instances (both CT and MR). In this case, for Tumor 1, there are two volume measurements (30 and 38.5) and the derived value is 34.25 (the average of the two). For Tumor 2, the situation is simpler, since the MR task did not contribute a volume instance, the system takes the instance from CT (30) and returns 30 for its derived value as well. System 10 uses a determined measurement value (e.g., tumor volume) in automatically generating report text that contains a descriptive phrase (e.g., “the tumor is enlarged” for volumes greater than 20 mm3).

System 10 generates a composite multi-modality imaging report including results derived from different imaging modality systems advantageously eliminating a need to generate separate CT and MR reports, for example, that a physician needs to manually interpret and edit. System 10 advantageously reports data from multiple different types of imaging modality device and merges the data into a single data model (identifying and resolving conflicts automatically). The system automatically accesses data from different types of imaging modality device in a multi-modality task workflow and produces multiple different reports that are targeted to a particular worker role (referring physician, nurse, surgeon). Individual reports contain measurement data derived from images produced by multiple different types of imaging modality in a task workflow. The system enables multi-modality imaging workflows to be configured for use at a care facility. The output of a workflow is a DICOM SR (or another type of evidence document format) that a user can map to be compatible with common reporting data model 203. A diagnosing physician employs Common Data Service system 250 to initiate a single reporting task that is capable of reporting on the data from the different types of imaging modality device tasks in a workflow.

A display image presented on display 19 (FIG. 1) enables a user to initiate generation of multiple reports targeted at different individuals which contain data from an individual type of imaging modality device or from multiple different types of imaging modality device 25. System 10 advantageously produces automated reports based on data from multiple modality tasks associated with corresponding multiple different types of imaging modality device by mapping data from evidence documents to common data model 203 (FIG. 2) compatible change data sets. Common data service system 250 converts from a change data set compatible with DICOM SR to an evidence document (or clinical finding) compatible Common Data Model 203. In one embodiment, Common Data Model 203 receives clinical finding changes from modality tasks as well as evidence document changes and uses XML files to convert from a DICOM SR based Evidence Document to a common data model change set. System 10 stores a unique clinical finding identifier (ID) in a memento area in Common Data Model 203. If a reporting task makes a change to data associated with (or initiated) by a particular imaging device type modality task, system 10 uses the memento stored in model 203 to map backward to a clinical finding and maps the clinical finding to an evidence document location and transmits the changed data back to the clinical task and updates the document location.

FIG. 9 shows a flowchart of a process performed by system 10 for processing medical report data associated with different types of imaging modality devices to provide a composite examination report. The different types of imaging modality device include at least two of, an MR imaging device, a CT scan imaging device, an X-ray imaging device, an Ultra-sound imaging device and a nuclear PET scanning imaging device. In step 812 following the start at step 811, acquisition processor 15 (FIG. 1) acquires multi-modality medical imaging examination report data for examinations performed on a patient by different types of imaging modality device. Report processor 29 in step 815 processes acquired multi-modality medical imaging examination report data items by selecting between individual data items in the acquired multi-modality medical imaging examination report data items to provide a single individual data item for incorporation in a composite report, in response to predetermined selection rules.

In step 817, report processor 29 resolves conflicts between the individual data items in the acquired multi-modality medical imaging examination report data items using the predetermined selection rules to select an individual data item in response to a predetermined accuracy hierarchy. The accuracy hierarchy ranks different imaging modality device type accuracy for a particular type of data item. In step 819, conversion processor 211 in common data service system 250 in report processor 29 converts the acquired multi-modality medical imaging examination report data having a first data format to converted data having a second format compatible with common data model 203 data and stores the converted data in the common data model 203 structure. Conversion processor 211 adaptively selects a converter from multiple different converters 213, 215 and 217 to convert the acquired multi-modality medical imaging examination report data, in response to Metadata associated with the acquired multi-modality medical imaging examination report data. The Metadata identifies a type of the first data format comprising at least one of, (a) a DICOM SR compatible data format, (b) an XML data format and (c) a proprietary data format. Conversion processor 211 also converts multi-modality medical imaging examination report data items retrieved from the common data model to be compatible with a particular imaging modality device using predetermined terms codes and identifiers stored in clinical dictionary 227.

In step 821, report processor 29 maps individual data items including the single individual data item in the acquired multi-modality medical imaging examination report data items, to corresponding data fields in a composite report data structure in memory in response to predetermined mapping information. Report processor 29 in step 823 processes the acquired multi-modality medical imaging examination report data items by merging data items from a first examination report associated with a first imaging modality device type with data items from a second examination report associated with a second imaging modality device type different to the first type. Report processor 29 processes acquired multi-modality medical imaging examination report data items retrieved from common data model 203 to provide the composite report.

In step 825, a configuration processor 220 in common data service system 250 initializes the system. Specifically, configuration processor 220 initializes clinical dictionary 227 by storing in dictionary 227 predetermined terms, codes or identifiers for use in data format conversion. Configuration processor 227 also initializes the conversion processor by storing in mapping processor 211 predetermined conversion data for converting at least one of, (a) a DICOM SR compatible data format, (b) an XML data format and (c) a proprietary data format, both to and from, a format used by common data model 203. In one embodiment Mapping processor 211 adaptively configures a conversion processor to convert the acquired multi-modality medical imaging examination report data to be compatible with a structure of common data model 203 and/or the composite report, in response to Metadata associated with the acquired multi-modality medical imaging examination report data. The Metadata identifies a format type of the medical imaging examination report data and/or the report data. In step 829 output processor 30 outputs data representing the composite report to a destination device. The process of FIG. 9 terminates at step 831.

A processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a controller or microprocessor, for example, and is conditioned using executable instructions to perform special purpose functions not performed by a general purpose computer. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A user interface (UI), as used herein, comprises one or more display images, generated by a user interface processor and enabling user interaction with a processor or other device and associated data acquisition and processing functions.

The UI also includes an executable procedure or executable application. The executable procedure or executable application conditions the user interface processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

A workflow processor, as employed by report processor 29, processes data to determine tasks to add to a task list, remove from a task list or modifies tasks incorporated on, or for incorporation on, a task list. A task list is a list of tasks for performance by a worker or device or a combination of both. A workflow processor may or may not employ a workflow engine. A workflow engine, as used herein, is a processor executing in response to predetermined process definitions that implement processes responsive to events and event associated data. The workflow engine implements processes in sequence and/or concurrently, responsive to event associated data to determine tasks for performance by a device and or worker and for updating task lists of a device and a worker to include determined tasks. A process definition is definable by a user and comprises a sequence of process steps including one or more, of start, wait, decision and task allocation steps for performance by a device and or worker, for example. An event is an occurrence affecting operation of a process implemented using a process definition. The workflow engine includes a process definition function that allows users to define a process that is to be followed and includes an Event Monitor, which captures events occurring in a Healthcare Information System. A processor in the workflow engine tracks which processes are running, for which patients, and what step needs to be executed next, according to a process definition and includes a procedure for notifying clinicians of a task to be performed, through their worklists (task lists) and a procedure for allocating and assigning tasks to specific users or specific teams. A document or record comprises a compilation of data in electronic form and is the equivalent of a paper document and may comprise a single, self-contained unit of information.

The system and processes of FIGS. 1-9 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. The system advantageously provides multiple automated reports from data produced by multiple different tasks associated with imaging a patient using different types of imaging modality device. Further, the processes and applications may, in alternative embodiments, be located on one or more (e.g., distributed) processing devices on the network of FIG. 1. Any of the functions and steps provided in FIGS. 1-9 may be implemented in hardware, software or a combination of both.

What is claimed is:

1. A system for processing medical report data associated with different types of imaging modality devices to provide a composite examination report, comprising:
an acquisition processor for acquiring multi-modality medical imaging examination report data for examinations performed on a patient by different types of imaging modality device;
a report processor for processing acquired multi-modality medical imaging examination report data items by,
(a) in response to predetermined selection rules, selecting between individual data items in the acquired multi-modality medical imaging examination report data items to provide a single individual data item for incorporation in a composite report and
(b) mapping individual data items including said single individual data item in the acquired multi-modality medical imaging examination report data items to corresponding data fields in a composite report data structure in memory in response to predetermined mapping information; and
an output processor for outputting data representing said composite report to a destination device.

2. A system according to claim 1, wherein
said report processor resolves conflicts between said individual data items in the acquired multi-modality medical imaging examination report data items using said predetermined selection rules to select an individual data item in response to a predetermined accuracy hierarchy, said accuracy hierarchy ranking different imaging modality device type accuracy for a particular type of data item.

3. A system according to claim 1, wherein
said report processor processes the acquired multi-modality medical imaging examination report data items by merging data items from a first examination report associated with a first imaging modality device type with data items from a second examination report associated with a second imaging modality device type different to said first type.

4. A system according to claim 1, wherein
said different types of imaging modality device include at least two of, (a) an MR imaging device, (b) a CT scan imaging device, (c) an X-ray imaging device, (d) an Ultra-sound imaging device and (e) a nuclear PET scanning imaging device.

5. A system according to claim 1, including
a conversion processor for converting the acquired multi-modality medical imaging examination report data having a first data format to converted data having a second format compatible with a common data model data and storing the converted data in the common data model structure wherein
said report processor processes acquired multi-modality medical imaging examination report data items retrieved from the common data model to provide said composite report.

6. A system according to claim 5, wherein
said conversion processor adaptively selects a converter from a plurality of different converters to convert the acquired multi-modality medical imaging examination report data, in response to Metadata associated with the acquired multi-modality medical imaging examination report data, said Metadata identifying a type of said first data format.

7. A system according to claim 6, wherein
said type of said first data format comprises at least one of, (a) a DICOM SR compatible data format, (b) an XML data format and (c) a proprietary data format.

8. A system according to claim 5, wherein
said conversion processor converts multi-modality medical imaging examination report data items retrieved from the common data model to be compatible with a particular imaging modality device using predetermined terms codes and identifiers stored in a clinical dictionary.

9. A system according to claim 8, including
a configuration processor for initializing said clinical dictionary by storing in said dictionary predetermined terms, codes or identifiers for use in data format conversion.

10. A system according to claim 5, including
a configuration processor for initializing said conversion processor by storing in said mapping processor predetermined conversion data for at least one of, (a) a DICOM SR compatible data format, (b) an XML data format and (c) a proprietary data format.

11. A system for processing medical report data associated with different types of imaging modality devices to provide a composite examination report, comprising:
an acquisition processor for acquiring multi-modality medical imaging examination report data for examinations performed on a patient by different types of imaging modality device;
a report processor for processing acquired multi-modality medical imaging examination report data items by,
(a) resolving conflicts between individual data items in the acquired multi-modality medical imaging examination report data items using predetermined selection rules to select an individual data item for incorporation in a composite report in response to a predetermined accuracy hierarchy, said accuracy hierarchy ranking different imaging modality device type accuracy for a particular type of data item and
(b) mapping individual data items including said individual data item in the acquired multi-modality medical imaging examination report data items to corresponding data fields in a composite report data structure in memory in response to predetermined mapping information; and
an output processor for outputting data representing said composite report to a destination device.

12. A system according to claim 11, including
said mapping processor adaptively configures a conversion processor to convert the acquired multi-modality medical imaging examination report data to be compatible with a structure of said composite report, in response to Metadata associated with the acquired multi-modality medical imaging examination report data, said Metadata identifying a format type of said report data.

13. A system according to claim 11, including
said mapping processor adaptively configures a conversion processor to convert the acquired multi-modality medical imaging examination report data to be compatible with a structure of a common data model, in response to Metadata associated with the acquired multi-modality medical imaging examination report data, said Metadata identifying a format type of said medical imaging examination report data and
said report processor processes acquired multi-modality medical imaging examination report data items retrieved from the common data model to provide said composite report.

14. A system according to claim 11, including
said report processor, in response to predetermined selection rules, selects between individual data items in the acquired multi-modality medical imaging examination report data items to provide a single individual data item for incorporation in said composite report.

15. A method for processing medical report data associated with different types of imaging modality devices to provide a composite examination report, comprising the activities of:
acquiring multi-modality medical imaging examination report data for examinations performed on a patient by different types of imaging modality device:
resolving conflicts between individual data items in the acquired multi-modality medical imaging examination report data items using predetermined selection rules to select an individual data item for incorporation in a composite report in response to a predetermined accuracy hierarchy, said accuracy hierarchy ranking different imaging modality device type accuracy for a particular type of data item;
mapping individual data items including said individual data item in the acquired multi-modality medical imaging examination report data items to corresponding data fields in a composite report data structure in memory in response to predetermined mapping information; and
outputting data representing said composite report to a destination device.

16. A method according to claim 15, including
merging data items including said individual data item from a first examination report associated with a first imaging modality device type with data items from a second examination report associated with a second imaging modality device type different to said first type.

17. A method for processing medical report data associated with different types of imaging modality devices to provide a composite examination report, comprising the activities of:
merging data items from a first examination report associated with a first imaging modality device type with data items from a second examination report associated with a second imaging modality device type different to said first type to provide a composite report by resolving conflicts between individual data items in the first and second examination reports using predetermined selection rules to select an individual data item for incorporation in a composite report in response to a predetermined accuracy hierarchy, said accuracy hierarchy ranking different imaging modality device type accuracy for a particular type of data item;
mapping said individual data item to a data field in said composite report data structure in memory in response to predetermined mapping information; and
outputting data representing said composite report to a destination device.
